(19) United States
(12) Patent Application Publication
(10) Pub. No.: US 2024/0139433 A1
(43) Pub. Date: May 2, 2024

(54) VASCULAR ACCESS SYSTEM AND METHOD FOR CONTINUOUS ULTRASOUND MONITORING

(71) Applicant: Becton, Dickinson and Company, Franklin Lakes, NJ (US)

(72) Inventors: Jonathan Karl Burkholz, Salt Lake City, UT (US); Matthew Prince, Herriman, UT (US)

(21) Appl. No.: 18/372,538

(22) Filed: Sep. 25, 2023

Related U.S. Application Data
(60) Provisional application No. 63/419,973, filed on Oct. 27, 2022.

(51) Int. Cl. A61M 5/42

(52) U.S. Cl. CPC A61M 5/427; A61M 2025/0166

(57) ABSTRACT

Vascular access systems and methods are provided for ultrasound monitoring. A vascular access system can include a vascular access device, an ultrasound assembly, a securement dressing, a base unit, and one or more monitoring devices. The ultrasound assembly can include an ultrasound probe that can be positioned overtop a catheter that is inserted into a patient's vasculature. Images from the ultrasound probe can be processed to generate parameters representing status, events, or other information about the catheter. Display content including the images and the parameters can be provided to the monitoring devices.

### RELATED APPLICATIONS

This application claims the benefit of U.S. Provisional Patent Application No. 63/419,973, which was filed on Oct. 27, 2022, which is incorporated herein in its entirety.

### BACKGROUND

Catheters are commonly used for a variety of infusion therapies. For example, catheters may be used for infusing therapeutic agents or fluids into a patient. Catheters may also be used for withdrawing blood from the patient. There are a variety of catheters commonly used in a medical setting, including, for example, peripherally-inserted central catheters, midline catheters, central venous catheters, dialysis catheters, and arterial catheters. A common type of catheter device includes a catheter that is over-the-needle. As its name implies, the catheter that is over-the-needle may be mounted over an introducer needle having a sharp distal tip.

The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. To verify proper placement of the introducer needle and/or the catheter in the blood vessel, a clinician generally confirms that there is “flashback” of blood in a flashback chamber of the catheter assembly. Once placement of the needle has been confirmed, the catheter may be left in place for future blood withdrawal or fluid infusion.

Although catheter indwell performance (i.e., how long the catheter can be safely left in the vasculature) has improved in recent years, there remains a significant number of complications that may develop throughout the intended dwell time of a vascular access device. These complications may include dislodgement, infiltration, extravasation, phlebitis, catheter-related infection, and loss of patency, among others.

FIG. 1 illustrates a prior art piezoelectric transducer array for performing ultrasound 10 (or ultrasound probe 10). Ultrasound probe 10 is conformable and can therefore be worn on the skin and used to perform deep tissue imaging.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to vascular access systems and methods for continuous ultrasound monitoring. A vascular access system can include a vascular access device, an ultrasound assembly, a securement dressing, a base unit, and one or more monitoring devices. The ultrasound assembly can include an ultrasound probe that can be positioned overtop a catheter that is inserted into a patient's vasculature. Images from the ultrasound probe can be processed to generate parameters representing status, events, or other information about the catheter. Display content including the images and the parameters can be provided to the monitoring devices.

In some embodiments, a vascular access system may include a vascular access device comprising a catheter, an ultrasound assembly comprising an ultrasound probe that is configured to be positioned overtop the catheter when the catheter is inserted into a patient's vasculature, and a base unit that is configured to receive images from the ultrasound probe.

In some embodiments, the ultrasound assembly may include a cable that connects the ultrasound probe to the base unit.

In some embodiments, the ultrasound assembly may include a wireless adapter that wirelessly connects the ultrasound probe to the base unit.

In some embodiments, the vascular access system may include a securement dressing that is configured to secure the ultrasound probe overtop the catheter.

In some embodiments, the base unit may be configured to generate display content from the images and transmit the display content to one or more monitoring devices.

In some embodiments, the base unit may be configured to generate one or more parameters from the images and to include the one or more parameters in the display content.

In some embodiments, the parameters may include catheter geometry or position information.

In some embodiments, the parameters may include one or more of catheter movement or displacement, catheter kinking, dislodgment events, extravasation, infiltration detection, thrombus development, phlebitis, patency indicators, blood flow characteristics, fluid administration flow characteristics, procedural events, or line draw tubing, probe or sensor position.

In some embodiments, a method for using a vascular access system includes inserting a catheter of a vascular access device into a patient's vasculature, positioning an ultrasound probe of an ultrasound assembly on the patient's skin overtop a portion of the catheter that is inserted into the patient's vasculature, and establishing a connection between the ultrasound probe and a base unit to thereby enable the base unit to receive ultrasound images from the ultrasound probe where the ultrasound images capture the portion of the catheter that is inserted into the patient's vasculature.

In some embodiments, the connection is wired or wireless.

In some embodiments, the method may also include placing a securement dressing overtop the ultrasound probe and a catheter adapter from which the catheter extends.

In some embodiments, a method for monitoring a catheter may include receiving, from an ultrasound probe that is positioned overtop a catheter that is inserted into a patient's vasculature, images that capture the catheter, processing the images to determine one or more parameters, and generating display content that includes the one or more parameters.

In some embodiments, processing the images to determine one or more parameters may include determining geometry or position information or the catheter, line draw tubing, a probe or a sensor.

In some embodiments, processing the images to determine one or more parameters may include detecting catheter movement or displacement.

In some embodiments, processing the images to determine one or more parameters may include detecting extravasation or infiltration.

In some embodiments, processing the images to determine one or more parameters may include detecting thrombus or phlebitis.

In some embodiments, processing the images to determine one or more parameters may include determining blood or fluid administration flow characteristics.

In some embodiments, processing the images to determine one or more parameters may include detecting a procedural event.

In some embodiments, the display content may also include at least some of the images.

In some embodiments, the one or more parameters may be determined using artificial intelligence.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 illustrates a prior art piezoelectric transducer array for performing ultrasound;

FIG. 2A provides an example of a vascular access system that is configured in accordance with one or more embodiments;

FIG. 2B provides another example of a vascular access system that is configured in accordance with one or more embodiments;

FIG. 3A is a partial cross-sectional view of a vascular access device that is configured in accordance with one or more embodiments when placed on the skin;

FIG. 3B is a transverse view of a vein in which a catheter is inserted that can be generated by a vascular access device that is configured in accordance with one or more embodiments;

FIG. 3C is a cross-sectional view of a vein in which a catheter is inserted that can be generated by a vascular access device that is configured in accordance with one or more embodiments;

FIG. 4 is an example display that can be generated by a vascular access system that is configured in accordance with one or more embodiments; and

FIG. 5 provides an example of electronic components that a base unit or monitoring device of a vascular access system may include in one or more embodiments.

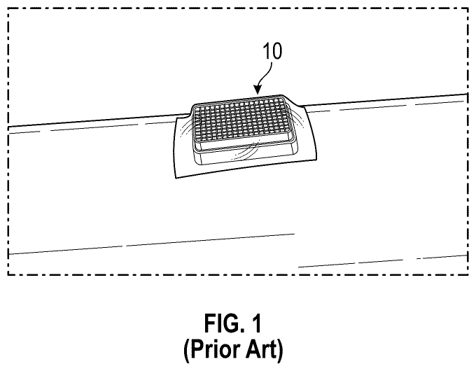

### DESCRIPTION OF EMBODIMENTS

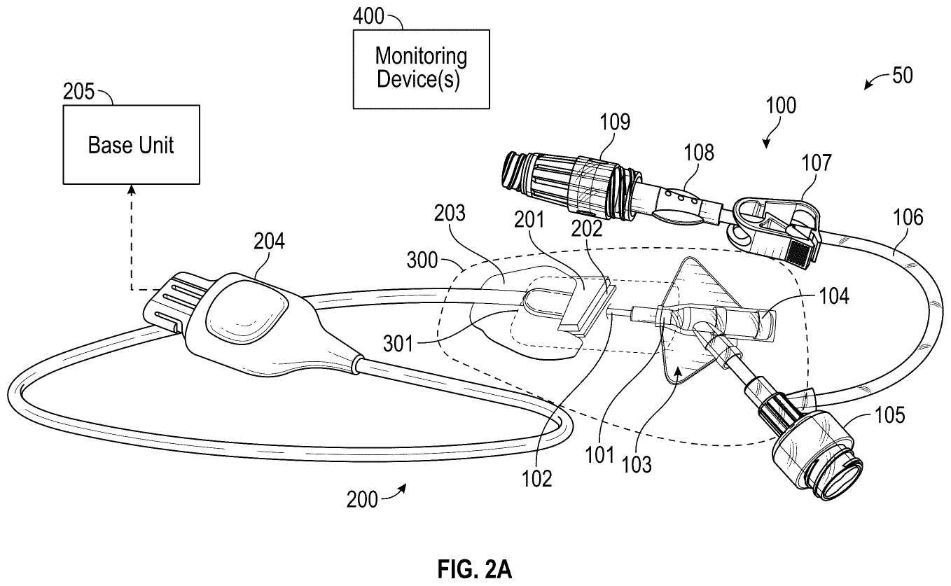

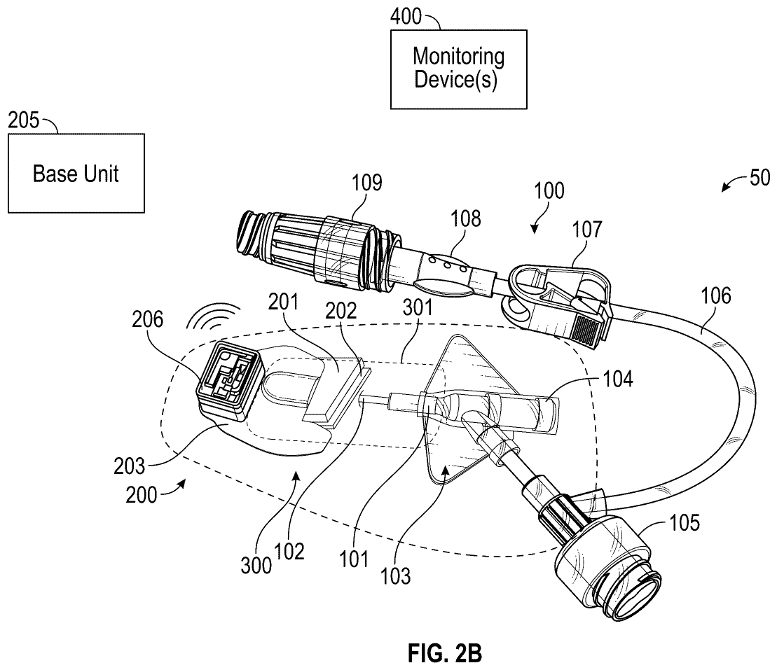

Referring now to FIG. 2A, in some embodiments, a vascular access system 50 may include a vascular access device 100, an ultrasound assembly 200, a securement dressing 300, and one or more monitoring devices 400. Vascular access device 100 may include a catheter adapter 101, a catheter 102 that extends distally from catheter adapter 101, a securement platform 103 (e.g., wings that extend outwardly from catheter adapter 101), a needle access port 104 by which an introducer needle (not shown) can be inserted through catheter adapter 101 and catheter 102, a near patient access port 105 which may be connected to a side port of catheter adapter 101, extension tubing 106 having a clamp 107 and to which luer adapter 108 is connected, and an access port 109 coupled to the luer adapter 108. This is only one example of various different configurations of vascular access devices that may be used in vascular access systems that are configured in accordance with embodiments of the present disclosure.

Ultrasound assembly 200 may include an ultrasound probe 201, a securing mechanism 202 for securing ultrasound probe 201 to the skin and/or to catheter 102, an electrical adapter 203 by which a cable 204 is connected to ultrasound probe 201, and a base unit 205 to which cable 204 may be connected to enable base unit 205 and ultrasound probe 201 to communicate. In some embodiments, securing mechanism 202 may be an adhesive film on the underside of ultrasound probe 201 that may be used to adhere ultrasound probe 201 directly to a patient's skin overtop catheter 102. In some embodiments, securing mechanism 202 can be a mechanical connection between ultrasound probe 201 and catheter adapter 101 and/or catheter 102. In some embodiments, electrical adapter 203 may be separable from ultrasound probe 201, while in other embodiments, electrical adapter 203 may be integrated with ultrasound probe 201. Base unit 205 can be any device that includes circuitry for communicating with ultrasound probe 201. In some embodiments, base unit 205 may provide power to ultrasound probe 201. In some embodiments, base unit 205 may directly process images received from ultrasound probe 201, while in other embodiments, base unit 205 may receive images from ultrasound probe 201 and forward the images to another device for processing. In some embodiments, base unit 205 may include user input elements to allow a user (e.g., a clinician and/or the patient) to control ultrasound probe 201. In some embodiments, base unit 205 may be connected to one or more other devices to allow users of the one or more other devices to control ultrasound probe 201.

Securement dressing 300 may be sized and shaped to cover and secure ultrasound probe 201 and catheter adapter 101 against the patient's skin. For example, in some embodiments, securement dressing 300 may fully cover catheter adapter 101 and may include an opening through which extension tubing 106 extends. The underside of securement dressing 300 may be adhesive to prevent securement dressing 300 from moving once placed on the skin. In some embodiments, a transparent window 301 may be formed in securement dressing 300 to facilitate viewing catheter 102 and ultrasound probe 201.

In some embodiments, ultrasound probe 201 may be integrated into securement dressing 300. In other embodiments, ultrasound probe 201 may be separate from securement dressing 300. In such embodiments, ultrasound probe 201 may be placed overtop catheter 102 and then securement dressing 300 may be placed overtop ultrasound probe 201 and catheter adapter 101. In any case, ultrasound probe 201 can be positioned on the patient's skin so that it is overtop the distal tip of catheter 102 when catheter 102 is inserted into the patient's vasculature.

Monitoring device(s) 400 can represent any device having a display on which images generated by ultrasound probe 201 may be displayed and/or on which information obtained from such images may be displayed. As examples, monitoring device(s) 400 could include a smart phone, a tablet, a laptop, a desktop, a thin client, a television, a dedicated display device, an infusion pump, a patient vital sign monitor, an arterial monitor, an ultrasound system visual display, etc. In some embodiments, a monitoring device 400 could function as base unit 205. A monitoring device 400 could also be configured to interface with one or more separate computing systems such as a system for storing patient data.

FIG. 2B provides another example configuration of vascular access system 50 in which a wireless adapter 206 in used in place of cable 204. Wireless adapter 206 may be configured to transmit images generated by ultrasound probe 201 to base unit 205 or possibly to monitoring device(s) 400. Wireless adapter 206 may also include batteries for powering ultrasound probe 201. In some embodiments, wireless adapter 206 may be integrated into electrical adapter 203, while in other embodiments, wireless adapter 206 may be selectively coupled to electrical adapter 203.

FIGS. 2A and 2B provide examples where vascular access system 50 includes a peripheral intravenous catheter. However, a vascular access system configured in accordance with embodiments of the present disclosure could be used with central venous catheters, peripherally inserted central catheters, midline catheters, arterial catheters, ports, venipuncture, sub-cutaneous access devices, or other indwelling tube, probe, sensor, or instrument.

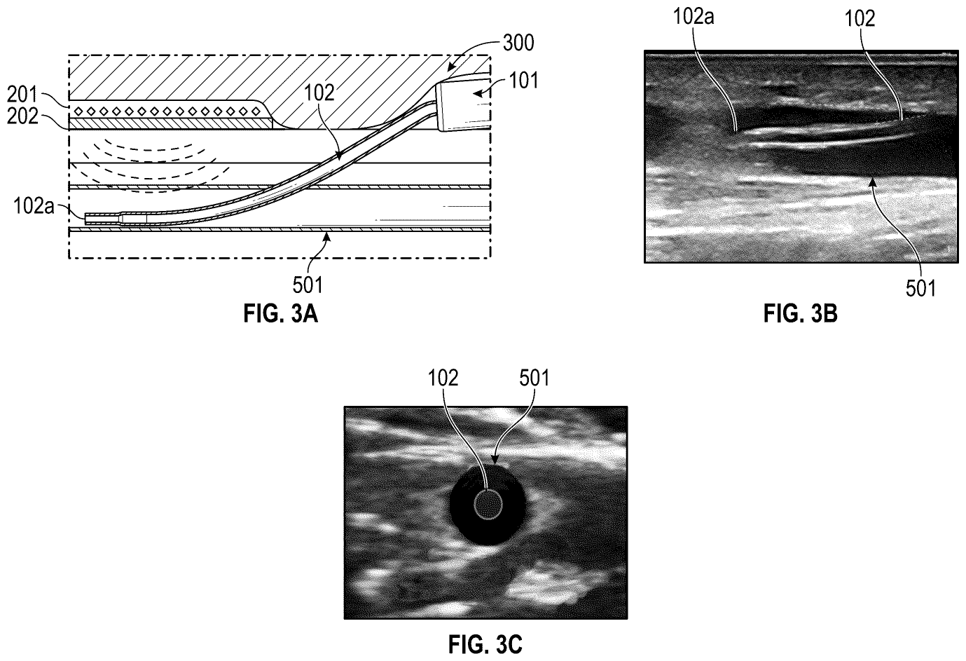

FIG. 3A is a partial cross-sectional view of vascular access system 50 when used on a patient. As shown, ultrasound probe 201 may be positioned via securing mechanism 202 on the patient's skin overtop a distal tip 102a of catheter 102 when catheter 102 is inserted into the patient's vasculature 501. Securement dressing 300 can be positioned overtop catheter adapter 101 and ultrasound probe 201 to retain this positioning of ultrasound probe 201 overtop distal tip 102a. In this position, ultrasound probe 201 can generate ultrasound images continuously, periodically, on demand, etc. of the portion of vasculature 501 within which catheter 102 extends including the surrounding tissue. For example, FIG. 3B is an image that captures a transverse view of vasculature 501, catheter 102, and distal tip 102a, and FIG. 3C is an image that captures a cross-sectional view of vasculature 501 and catheter 102.

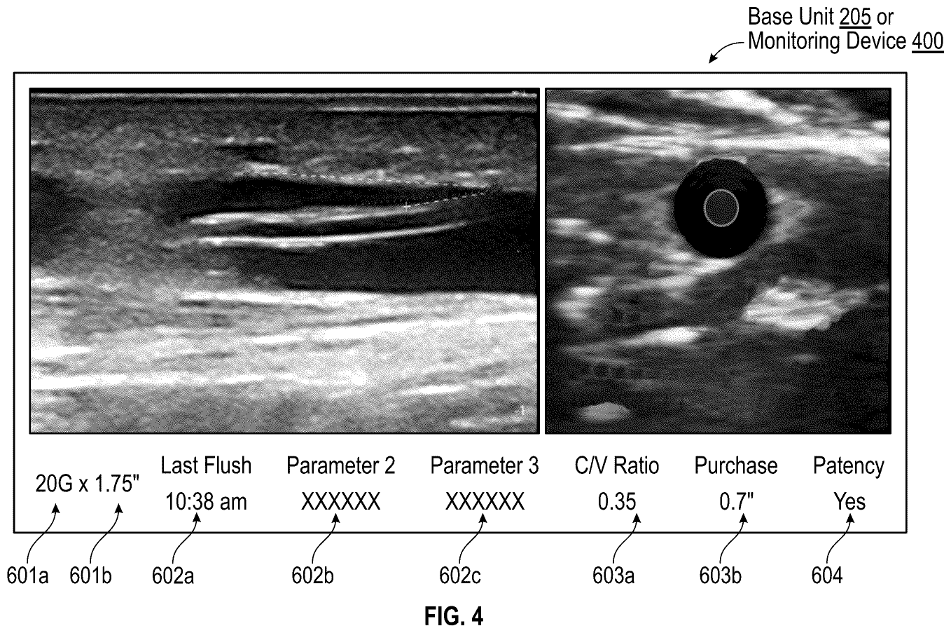

FIG. 4 provides an example of how images generated by ultrasound probe 201 can be integrated into a display along with various information derived from the images. As indicated, this display could be generated and/or presented on base unit 205 and/or any number of monitoring devices 400. This display may include one or more views of catheter 102 within vasculature 501 such as the transverse view of FIG. 3B and the cross-sectional view of FIG. 3C. The transverse view may allow a clinician to see how catheter 102 is extending into vasculature 501 and may therefore facilitate quickly determining if catheter 102 is inserted sufficiently, if distal tip 102a is positioned correctly, if there is any blockage, or any other condition that is capable of being detected via ultrasound. The cross-sectional view may allow a clinician to see how a particular portion of catheter 102 is positioned within vasculature 501 and may therefore facilitate quickly determining if catheter 102 may be excessively limiting blood flow through vasculature 501 or any other condition that is capable of being detected via ultrasound. In some embodiments, a user may be able to adjust the location of the views generated by ultrasound probe 201. For example, a user may be able to move the cross-sectional view along the length of catheter 102 to determine if there is excessive blockage at any portion along the length of catheter 102.

FIG. 4 also illustrates that the display may include a variety of information that may be derived from the images that ultrasound probe 201 produces or from input. For example, the display includes an indicator 601a of the gauge of catheter 102 and an indicator 601b of the length of catheter 102. Indicators 601a and 601b could be obtained via user input or could be calculated from the images produced by ultrasound probe 201.

The display also includes indicators 602a, 602b, and 602c for different parameters. In some embodiments, these parameters could be selectable. For example, in FIG. 4, indicator 602a provides information for when catheter 102 was last flushed. This last flush information could be calculated using the images produced by ultrasound probe 201. For example, doppler techniques could be applied to the image data to detect when fluid is flowing out through distal tip 102a, and in response to such a detection, base unit 205 (or a monitoring device 400) could store an indication that a flush has occurred at that time. In FIG. 4, indicators 602b and 602c have not been selected. However, these indicators could be selected to display information for any of many different conditions, events, statuses, etc. as described below.

The display further includes indicators 603a and 603b that provide information about the portion of catheter 102 that is inside vasculature 501. Indicator 603a defines the catheter to vein ratio (i.e., the ratio of the catheter's diameter to the vein's diameter at a particular location). Indicator 603b defines the purchase of catheter 102 (i.e., the length of catheter 102 that is inside vasculature 501). The display additionally includes an indicator 604 defining a patency status of catheter 102 (i.e., whether catheter 102 can safely remain within vasculature 501). Base unit 205 (or a monitoring device 400) could calculate the patency status using the images provided by ultrasound probe 201 (e.g., to detect the extent to which catheter 102 and/or vasculature 501 around catheter 102 may be blocked).

As suggested above, vascular access system 50 can be configured to monitor and/or display information relating to the status of catheter 102, vasculature 501, or the surrounding tissue and a variety of associated physiological or procedural parameters by leveraging images that are provided by ultrasound probe 201. This information includes catheter geometry information (e.g., the catheter to vein ratio, the purchase of the catheter, flow restrictions around the catheter), catheter position information (axial position of the catheter within the vein, the position or angle of the distal tip of the catheter relative a vein wall, valve, branch or other physiological feature), catheter movement or displacement, catheter kinking, dislodgment events, extravasation, infiltration detection (e.g., by monitoring tissue surrounding vasculature 501), thrombus development, phlebitis (visual or correlated cumulative movement), patency indicators, blood flow characteristics (e.g., by using doppler to detect velocity and/or volume of blood flowing into catheter 102), fluid administration flow characteristics (e.g., by using doppler to detect velocity, volume, direction, and/or duration of fluid flow), procedural events (e.g., flush, draw, fluid administration), and/or line draw tubing, probe or sensor position in the vein or relative to the distal tip of the catheter or physiological feature (e.g., thrombus, valve, wall, branch, etc.).

Vascular access system 50 may provide a display including indicators of any of the above-mentioned information and may provide corresponding alerts. For example, base unit 205 or a monitoring device 400 may be configured to output a visual, audible, tactile, or digital alert when a condition or event is detected from the ultrasound images. In some embodiments, ultrasound probe 201, electrical adapter 203, cable 204, and/or wireless adapter 206 could include one or more alerting mechanisms (e.g., LEDs, speakers, haptic units, etc.) to provide an alert.

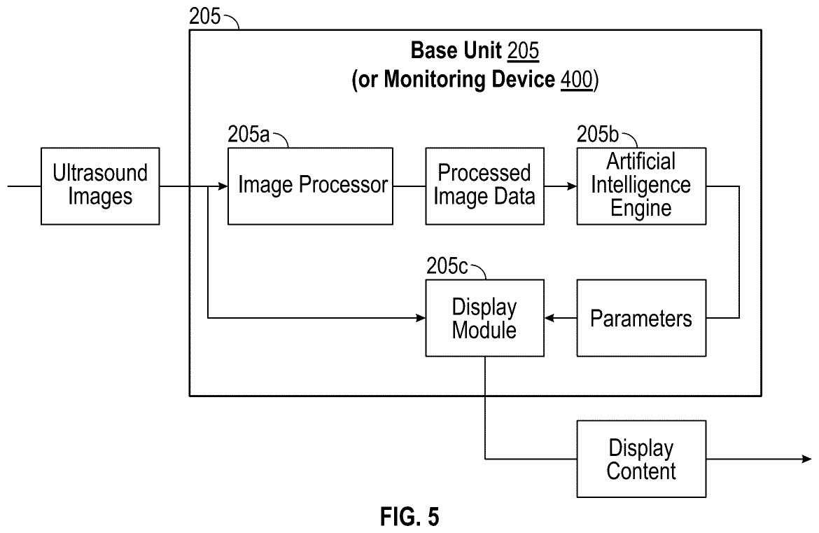

FIG. 5 provides an example of how base unit 205 (or possibly monitoring device 400) could be configured to generate display content from ultrasound images. This display content can include any of the above-described information, indicators, status, events, alerts, etc. (collectively “parameters”). FIG. 4 is one example of display content.

Base unit 205 may be configured to receive ultrasound images from ultrasound probe 201 continuously, periodically, on demand, etc. Base unit 205 may include an image processor 205a that is configured to process the ultrasound images to generate processing image data. This processed image data can be input to an artificial intelligence engine 205b that may be configured to detect and/or generate parameters from the processed image data. The parameters along with the ultrasound images can be provided to a display module 205c that can generate the display content that includes the images and the parameters.

In some embodiments, image processor 205a can be configured to determine from an image or sequence of images various status information such as catheter geometry or position information or the presence of a thrombus, kink, or other blockage. In some embodiments, artificial intelligence engine 205b can be trained to detect when parameters are present in a stream of ultrasound images. For example, artificial intelligence engine 205b could detect when a sequence of ultrasound images is indicative of a flush event, a draw event, the occurrence of extravasation, a dislodgement or movement event, etc.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A vascular access system comprising:
a vascular access device comprising a catheter;
an ultrasound assembly comprising an ultrasound probe that is configured to be positioned overtop the catheter when the catheter is inserted into a patient's vasculature; and
a base unit that is configured to receive images from the ultrasound probe.

2. The vascular access system of claim 1, wherein the ultrasound assembly includes a cable that connects the ultrasound probe to the base unit.

3. The vascular access system of claim 1, wherein the ultrasound assembly includes a wireless adapter that wirelessly connects the ultrasound probe to the base unit.

4. The vascular access system of claim 1, further comprising:
a securement dressing that is configured to secure the ultrasound probe overtop the catheter.

5. The vascular access system of claim 1, wherein the base unit is configured to generate display content from the images and transmit the display content to one or more monitoring devices.

6. The vascular access system of claim 5, wherein the base unit is configured to generate one or more parameters from the images and to include the one or more parameters in the display content.

7. The vascular access system of claim 6, wherein the parameters include catheter geometry or position information.

8. The vascular access system of claim 6, wherein the parameters include one or more of catheter movement or displacement, catheter kinking, dislodgment events, extravasation, infiltration detection, thrombus development, phlebitis, patency indicators, blood flow characteristics, fluid administration flow characteristics, procedural events, or line draw tubing, probe or sensor position.

9. A method for using a vascular access system comprising:
inserting a catheter of a vascular access device into a patient's vasculature;
positioning an ultrasound probe of an ultrasound assembly on the patient's skin overtop a portion of the catheter that is inserted into the patient's vasculature; and
establishing a connection between the ultrasound probe and a base unit to thereby enable the base unit to receive ultrasound images from the ultrasound probe where the ultrasound images capture the portion of the catheter that is inserted into the patient's vasculature.

10. The method of claim 9, wherein the connection is wired or wireless.

11. The method of claim 9, further comprising:
placing a securement dressing overtop the ultrasound probe and a catheter adapter from which the catheter extends.

12. A method for monitoring a catheter comprising:
receiving, from an ultrasound probe that is positioned overtop a catheter that is inserted into a patient's vasculature, images that capture the catheter;
processing the images to determine one or more parameters; and
generating display content that includes the one or more parameters.

13. The method of claim 12, wherein processing the images to determine one or more parameters comprises determining geometry or position information or the catheter, line draw tubing, a probe or a sensor.

14. The method of claim 12, wherein processing the images to determine one or more parameters comprises detecting catheter movement or displacement.

15. The method of claim 12, wherein processing the images to determine one or more parameters comprises detecting extravasation or infiltration.

16. The method of claim 12, wherein processing the images to determine one or more parameters comprises detecting thrombus or phlebitis.

17. The method of claim 12, wherein processing the images to determine one or more parameters comprises determining blood or fluid administration flow characteristics.

18. The method of claim 12, wherein processing the images to determine one or more parameters comprises detecting a procedural event.

19. The method of claim 12, wherein the display content also includes at least some of the images.

20. The method of claim 12, wherein the one or more parameters are determined using artificial intelligence.
